(19) United States
(12) Patent Application Publication
(10) Pub. No.: US 2025/0340810 A1
(43) Pub. Date: Nov. 6, 2025

(54) METHOD, SYSTEM AND APPARATUS FOR DEPLOYING A FLEXIBLE BIOPROCESSING VESSEL

(71) Applicant: Global Life Sciences Solutions USA LLC, Marlborough, MA (US)

(72) Inventor: Matthew Ouellette, Marlborough, MA (US)

(73) Assignee: Global Life Sciences Solutions USA LLC, Marlborough, MA (US)

(21) Appl. No.: 18/654,346

(22) Filed: May 3, 2024

(51) Int. Cl. C12M 1/00

(52) U.S. Cl. CPC C12M 23/50; C12M 23/14; C12M 23/28; C12M 23/52

(57) ABSTRACT

A method for deploying a flexible bioprocessing vessel includes placing a flexible bioprocessing vessel within a rigid support structure, the flexible bioprocessing vessel having a plurality of circumferentially arranged hoisting points arranged around an upper portion of the flexible bioprocessing vessel. The method further includes connecting the plurality of circumferentially arranged hoisting points to a vessel hoist mechanism, inflating the flexible bioprocessing vessel, and hoisting the flexible bioprocessing vessel within the rigid support structure, while it is inflating, via the plurality of circumferentially arranged hoisting points and vessel hoist mechanism until it is deployed within the rigid support structure. Wherein the plurality of circumferentially arranged hoisting points reduces a possibility of undesirable wrinkling of the flexible bioprocessing vessel during deployment.

### BACKGROUND

### Technical Field

Embodiments of the invention relate generally to bioprocessing, and, more particularly, to a method and system for deploying a flexible bioprocessing vessel within a rigid support structure.

### Discussion of Art

Mixers and bioreactors are often used to carry out biochemical and biological processes and/or manipulate liquids and other products of such processes. These devices typically utilize single-use vessels, e.g., flexible or collapsible bags, that are supported by an outer rigid structure such as a stainless-steel housing/tank.

In use, a disposable/single-use bag is positioned within the rigid tank and filled with the desired fluid for processing. An impeller assembly that includes a rotating impeller having one or more blades is disposed within the bag and is used to mix the fluid. Existing impeller systems are either top-driven, having a shaft that extends downwardly into the bag, on which one or more impellers are mounted, or bottom-driven, having an impeller disposed in the bottom of the bag that is driven by, for example, a magnetic drive system positioned outside the bag.

Rigid support structures, e.g., stainless steel tanks, may be relatively large, having capacities of 2000 L-3000 L or more. As will be appreciated, 2000 L and 3000 L flexible bags configured for use within such support structures are likewise relatively large and given their size and flexible nature, may be challenging install within such structures. Many known bags, particularly larger capacity bags, may require mechanical assistance when being deployed within a rigid base. In particular, a powered mechanical hoist mechanism may be used along with air inflation to raise the vessel to its fully deployed state.

Known bags have hoist points which may assist in raising them into a deployed stated. However, these bags include relatively few hoist points (e.g., 2-4 such points). These hoist points are too few and/or improperly located to prevent or reduce the possibility of wrinkling of the flexible bag during deployment. Such wrinkling is undesirable as it creates localized areas within the bioprocess media that are not uniform with the rest of the bulk fluid, e.g. depleted oxygen zones. When installing bags into support structures, users try to minimize wrinkles and sometimes pull/manipulate bags in an effort to minimize these wrinkles. Such manipulation can increase the chance of accidentally compromising the bag.

It may also be desirable to utilize a flexible vessel/bag that has a hexagonal structure, e.g., a bag having eight flexible panels (six side panels, a top panel, and a bottom panel, that are welded together) within a cylindrical rigid support structure, e.g., tank. As will be appreciated, hoisting solutions for such bags are generally desirable, particularly those that prevent or reduce the probability of wrinkling as noted above.

In view of the above, there is a need for a system and method for deploying a flexible bioprocessing vessel in a rigid cylindrical support structure.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, a method for deploying a flexible bioprocessing vessel includes placing a flexible bioprocessing vessel within a rigid support structure, the flexible bioprocessing vessel having a plurality of circumferentially arranged hoisting points arranged around an upper portion of the flexible bioprocessing vessel. The method further includes connecting the plurality of circumferentially arranged hoisting points to a vessel hoist mechanism, inflating the flexible bioprocessing vessel, and hoisting the flexible bioprocessing vessel within the rigid support structure, while it is inflating, via the plurality of circumferentially arranged hoisting points and vessel hoist mechanism until it is deployed within the rigid support structure. Wherein the plurality of circumferentially arranged hoisting points reduces a possibility of undesirable wrinkling of the flexible bioprocessing vessel during deployment.

In an embodiment, the flexible bioprocessing vessel includes at least six side panels forming sides of the flexible bioprocessing vessel, a top panel adjoining the at least six side panels, the top panel forming a top of the flexible bioprocessing vessel, and a bottom panel adjoining the at least six side panels, the bottom panel forming a bottom of the flexible bioprocessing vessel, the at least six side panels, top panel, and bottom panel defining an interior cavity configured for processing a fluid, the at least six side panels joined together via vertical welded seams.

In an embodiment, the plurality of circumferentially arranged hoisting points are six circumferentially arranged hoisting points, each of the six circumferentially arranged hoisting points located proximate to a junction of one of the vertical welded seams and a seam connecting the top panel to the at least six side panels.

In an embodiment, the six circumferentially arranged hoisting points are film eyelet tabs.

In an embodiment, the flexible bioprocessing vessel further includes a central hoisting point positioned at an approximate mid-point of a top panel of the flexible bioprocessing vessel.

In an embodiment, the method further includes connecting probes, at least one fluid input, and/or at least one fluid output to the flexible bioprocessing vessel.

In an embodiment, the rigid support structure is a 2000 L tank having a cylindrical interior.

In another aspect of the invention, a flexible bioprocessing vessel includes at least four side panels forming sides of the flexible bioprocessing vessel, a top panel adjoining the at least four side panels, the top panel forming a top of the flexible bioprocessing vessel, a bottom panel adjoining the at least four side panels, the bottom panel forming a bottom of the flexible bioprocessing vessel, the at least four side panels, top panel, and bottom panel defining an interior cavity configured for processing a fluid, the at least four side panels joined together via vertical welded seams. The vessel includes at least one fluid input and at least one fluid output for adding and removing fluid to and from the interior cavity respectively and a plurality of circumferentially arranged hoisting points arranged around the top of the flexible bioprocessing vessel. Wherein the plurality of circumferentially arranged hoisting points reduces a possibility of undesirable wrinkling of the flexible bioprocessing vessel during deployment within a rigid support structure.

In an embodiment, the at least four side panels are six side panels.

In an embodiment, the vessel includes six side panels and the plurality of circumferentially arranged hoisting points are six circumferentially arranged hoisting points.

In an embodiment, the plurality of circumferentially arranged hoisting points are film eyelet tabs.

In an embodiment, the flexible bioprocessing vessel further includes a central hoisting point positioned at an approximate mid-point of the top panel of the flexible bioprocessing vessel.

In an embodiment, each of the at least four side panels are thermally welded to adjacent side panels.

In an embodiment, the at least four side panels are seven side panels.

In an embodiment, the at least four side panels are eight side panels.

In an embodiment, the flexible bioprocessing vessel has an overall height of about 2100 mm and an overall point to point width of about 1304 mm and is configured for use with a 2000 L rigid support structure having a cylindrical interior.

In another embodiment, the flexible bioprocessing vessel has an overall height of about 1350 mm and an overall point to point width of about 814 mm and is configured for use with a 500 L rigid support structure having a cylindrical interior.

According to another aspect of the invention, a system for bioprocessing includes a rigid support structure having a cylindrical interior that includes a bottom surface and a substantially open top, a vessel hoist mechanism, and a flexible bioprocessing vessel. The vessel includes at least four side panels forming sides of the flexible bioprocessing vessel, a top panel adjoining the at least four side panels, the top panel forming a top of the flexible bioprocessing vessel, a bottom panel adjoining the at least four side panels, the bottom panel forming a bottom of the flexible bioprocessing vessel, the at least four side panels, top panel, and bottom panel defining an interior cavity configured for processing a fluid, the at least four side panels joined together via vertical welded seams. The vessel further includes at least one fluid input and at least one fluid output for adding and removing fluid to and from the interior cavity respectively and a plurality of circumferentially arranged hoisting points arranged around the top of the flexible bioprocessing vessel and configured for attachment to the vessel hoist mechanism. Wherein the plurality of circumferentially arranged hoisting points reduces a possibility of undesirable wrinkling of the flexible bioprocessing vessel during deployment within the rigid support structure.

In an embodiment, the plurality of circumferentially arranged hoisting points are six circumferentially arranged hoisting points, each of the six circumferentially arranged hoisting points.

In an embodiment, the plurality of circumferentially arranged hoisting points are film eyelet tabs.

In another aspect of the invention, an apparatus for deploying a flexible bioprocessing vessel within a rigid support structure includes a motor connected to a motor support structure that extends over an open top of the rigid support structure. The apparatus further includes an attachment frame operatively connected to the motor, the attachment frame configured for attachment to a plurality of circumferentially arranged hoisting points arranged around a top panel of the flexible bioprocessing vessel. Wherein the apparatus is configured to hoist the flexible bioprocessing vessel within the rigid support structure, while it is inflating, via the plurality of circumferentially arranged hoisting points until it is deployed within the rigid support structure and wherein the apparatus and the plurality of circumferentially arranged hoisting points reduces a possibility of undesirable wrinkling of the flexible bioprocessing vessel during deployment.

In an embodiment the attachment frame includes a central member which is attached to a selectively retractable line of the motor and a hoist member formed on or attached to each distal end of the central member. The attachment frame further includes a plurality of downwardly depending legs located on each hoist member, the downwardly depending legs configured for connection to the plurality of circumferentially arranged hoisting points arranged around the top of the flexible bioprocessing vessel.

In an embodiment, the attachment frame includes a hub that includes a plurality of radially extending spokes that are configured for connection to the plurality of circumferentially arranged hoisting points arranged around the top of the flexible bioprocessing vessel.

### BRIEF DESCRIPTION OF THE FIGURES

The present invention will be better understood from reading the following description of non-limiting embodiments, with reference to the attached drawings, wherein below:

FIG. 1 is an image of a known flexible bioprocessing vessel and hoist mechanism depicting undesirable sagging of the vessel between hoist points;

FIG. 2 is a perspective view of a system for deploying a flexible bioprocessing vessel according to an embodiment of the invention;

FIG. 3 is a rear perspective view of the system of FIG. 2;

FIG. 4 is a perspective view of a system for deploying a flexible bioprocessing vessel according to an alternative embodiment of the invention;

FIG. 5 is a graphical illustration of an eight-panel flexible bioprocessing vessel for use with embodiments of the invention;

FIG. 6 is a top view of an eight-panel vessel deployed within a cylindrical rigid support structure; and

FIG. 7 is a perspective view of the vessel of FIG. 7 depicted a circumferentially arranged hoisting point according to an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioprocessing vessels having a wall or a portion of a wall that is flexible, single-use flexible bags, as well as other containers or conduits commonly used in biological or chemical processing, including, for example, cell culture/purification systems, fermentation systems, mixing systems, media/buffer preparation systems, and filtration/purification systems.

As used herein, the term “bag” means a flexible or semi-rigid vessel used, for example, as a mixer or bioreactor for the contents within.

Embodiments may be utilized in connection with a wide variety of biological and chemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. Certain embodiments may be suitable for use in other industries where installation and/or deployment of a flexible object within a rigid structure is desirable.

While certain embodiments may be suitable for use with 500 L and 2000 L bioprocessing vessels, other embodiments may be used with vessels of a variety of sizes, e.g., 3000 L and larger.

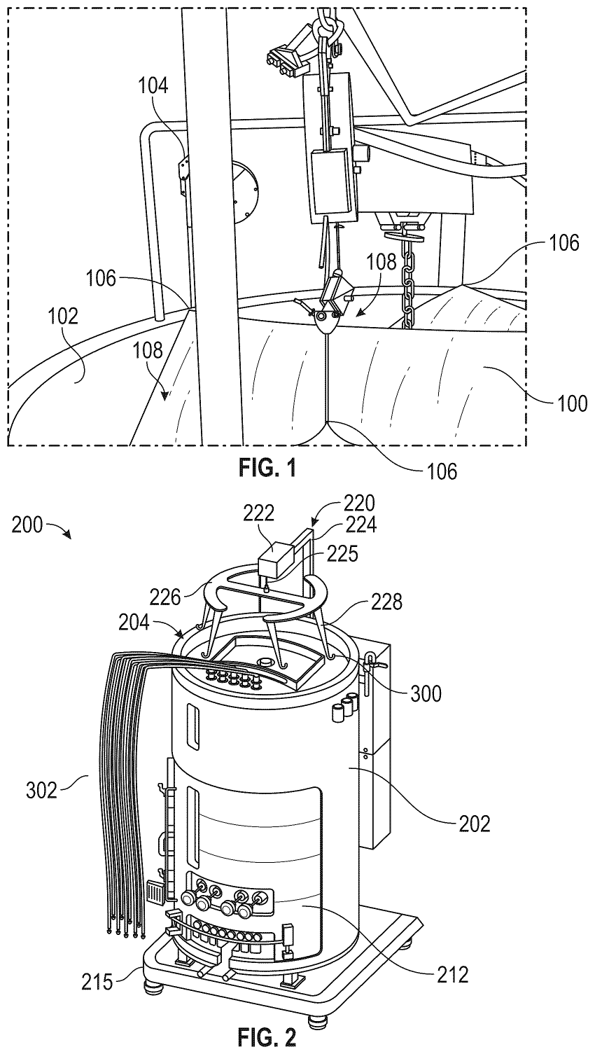

Referring to FIG. 1 a known flexible bioprocessing vessel, bag 100, and cylindrical tank/rigid support structure 102 are depicted. The bag 100 is shown in a deployed configuration within the structure. As shown, the bag 100 may be deployed within the support structure with the assistance of a hoisting mechanism that includes several mechanical spring assists (e.g., torque reels 104) as well as an electric motor/lift which may connect to a central attachment point on the vessel (not shown). In use, the torque reels 104 bear some of the weight of the film and help lift and guide the bag 100 as it is deployed within the tank. Generally, deployment of the vessel within the tank involves use of the aforementioned electric motor and/or inflation of the vessel with air.

As shown, however, the bag 100 has relatively few attachment points 106 that are disposed proximate to seams between panels near the outer perimeter of the bag. More specifically, known bags may include, e.g., four panels with four seams and four attachment points. These configurations of attachment points allow side walls of the bag to droop (see area 108) between adjacent points. This drooping results in undesirable wrinkling of the bag within the support structure.

Referring now to FIG. 2, a system for bioprocessing in accordance with an embodiment of the invention is depicted. The system includes a rigid support structure 200 includes a rigid body 202 having an interior 204. The interior 204, which is cylindrical, has an open top and a selectively openable door 212 which allows access to the interior 204, which is open and configured to receive a flexible bioprocessing vessel 300. The rigid support structure further includes a bottom surface on which the vessel sits during use. The rigid support structure 200 further includes a stand portion 215 attached to the rigid body 202 which allows for access to the space below the body.

As shown, the rigid support structure 200 further includes an apparatus for deploying a flexible bioprocessing vessel within the support structure, e.g., vessel hoist mechanism 220. The hoist mechanism includes a motor 222 which is attached to a motor support structure, e.g., arm 224, that extends over the open top 210 of the rigid body 202. The arm may be fixed to the support structure or may be located on a separate apparatus or structure adjacent to the rigid support structure 200. The motor includes a selectively retractable line 225 that is connected to an attachment frame 226 which includes a plurality of downwardly depending legs 228, which are connected to a plurality of circumferentially arranged hoisting points arranged around the top of the flexible bioprocessing vessel. In the depicted embodiment, there are six legs, each leg being connected to a hoisting point on the vessel.

In embodiments, there may be more than six circumferentially arranged hoisting points (and legs), e.g., seven or eight such points. In other embodiments, the vessel may have fewer sides than hoisting points. For example, a four-sided vessel may include six or more hoisting points arranged, e.g., evenly spaced, about its four side panels. Likewise, a five-sided vessel may include six or more hoisting points on its side panels to prevent the side panels from sagging and/or wrinkling during deployment. As such, in embodiments, the vessel has at least four side panels and a plurality of circumferentially arranged hoisting points.

In certain embodiments where the rigid support structure geometry matches or closely approximates that of the flexible vessel, the vessel may have the same number of hoisting points as side panels, e.g., four hoisting points for a four-sided vessel for use in a four-sided support structure. In such embodiments, where the vessel closely matches the geometry of the support structure, the probability and/or severity of wrinkling is reduced such that a hoist mechanism connected to fewer than six (e.g., four) hoisting points may be effective in deployment without significant wrinkling.

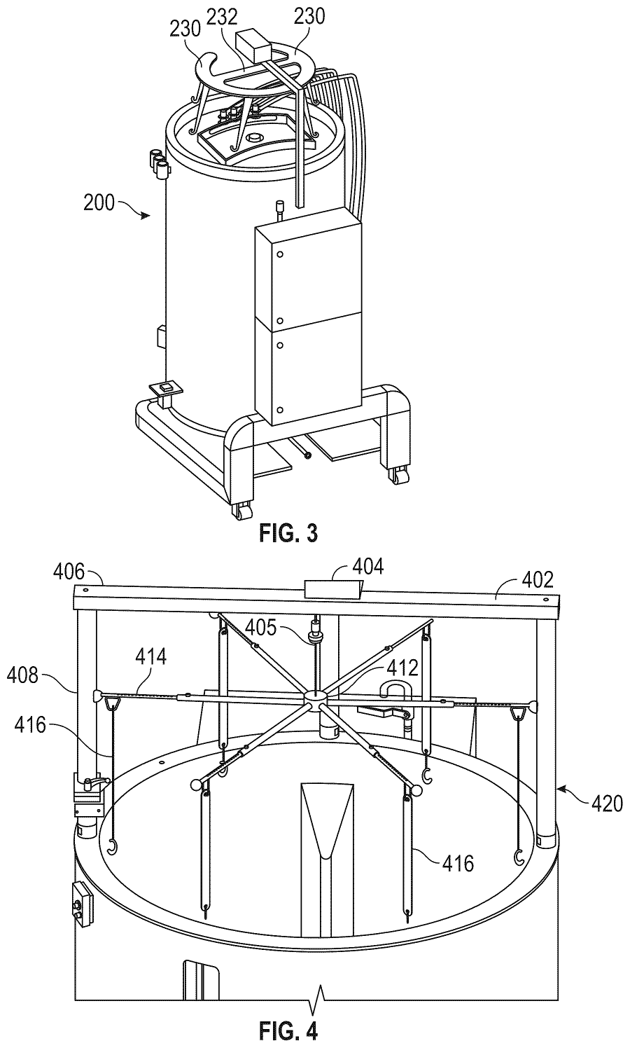

Referring to FIG. 2 and FIG. 3, in embodiments, the attachment frame 226 includes a central member 232 having opposed distal ends. The central member 232 may include an attachment point in substantially the center of the central member 232 to which the selectively retractable line 225 of the motor is attached. A curved or arcuate hoist member 230 is formed on or attached to each of the distal ends of the central member 232. Each hoist member has a radius of curvature that approximates or is complementary to a radius of curvature of the cylindrical support structure and/or the placement of circumferentially arranged hoisting points on the vessel. That said, in embodiments, the attachment frame 226 may be sized such that it may be lowered within the cylindrical interior of the rigid support structure.

As shown, each of the hoist members includes three downwardly depending legs that are uniformly spaced apart on the hoist member. As will be appreciated, in embodiments, the legs are spaced such that they are located substantially directly above a hoisting point on the vessel. In other embodiments, greater that three legs or fewer than three legs may be located on a hoist member.

In the depicted embodiment, each of the downwardly depending legs is connected to a hoisting points (e.g., a grommet or eyelet in the film of the vessel) via a wire or cable attached to a fastener such as a lanyard clip. In other embodiments, the legs may be directly connected to hoisting points. In certain other embodiments, the attachment frame 226 may be part of the vessel itself and may be selectively connectable to the hoist mechanism. In such embodiments the frame may be manufactured from a material suitable for a single use, e.g., a plastic. In certain embodiments, the attachment frame 226 may be expandable. That is the central member 232 may be slidably adjustable lengthwise so that the frame may be used with larger or smaller rigid support structures/tanks.

In embodiments, the attachment frame 226 is substantially open. That is, there are open spaces between the legs which allow tubing 302 to run from a vessel to equipment that is adjacent to or separate from the support structure.

Referring now to FIG. 4, an alternative embodiment of a vessel hoist mechanism is depicted. The hoist mechanism 420 includes a motor 404 which is attached to a motor support structure, e.g., cross member 406 that extends over the open top of the rigid body. The cross member 406 is secured to the rigid body via two vertical supports 408 that are attached to the rigid body. As will be appreciated, the vertical supports may be attached via fasteners or may be unitary with the support structure.

The motor 404 includes a selectively retractable line 405 that is connected to an attachment frame 411 which, in this embodiment, is a hub 412 that includes a plurality of radially extending spokes 414, e.g., six spokes. In certain embodiments, the spokes may be adjustable, e.g., telescoping, so that the hoist mechanism 420 may be used with cylindrical support structures of varying sizes. The spokes each include a connecting rod 416 that hangs from the spoke, the connecting rods having a first distal end which is connected to the spoke and a second distal end opposite the first, which is connected to the vessel. In embodiments, a length of flexible material such as a strap may be used in lieu of a connecting rod. The connecting rods are movable relative to the spokes and may be attached thereto via a hook/eyelet connection although other connections/fasteners may be employed without departing from the invention. The second distal end of each connecting rod is attached to a circumferential hoisting point on the vessel. In embodiments, there are six connecting rods configured for connection to six circumferentially arranged hoisting points. In other embodiments, more than six hoisting points (and connecting rods and spokes), e.g., seven or eight such points, may be employed.

In the embodiments of FIG. 2 and FIG. 4, a central hoisting point 604 may also be utilized in addition to the six circumferentially arrange hoisting points (FIG. 6). The central hoisting point may be positioned at an approximate mid-point of a top panel of the flexible bioprocessing vessel. This provides an additional lifting point to expedite the deployment process and further reduce wrinkling.

As will be appreciated, the vessel hoist mechanism may be manufactured from a variety of materials, e.g., metals, plastics, etc. The hoist motor may be a variety of motor types and powers, and the invention is not so limited. In embodiments, the vessel hoist mechanism may be configured for use with a foam detection system, e.g., a foam camera. The camera may be supported by or integrated into the attachment frame.

In embodiments, the hoist mechanism may remain connected to the vessel during use such that additional hoisting of the vessel necessitated by the processing occurring therein can be carried out. In other embodiments, the hoist mechanism can be disconnected and moved out of the space above the open top of the rigid support structure/tank.

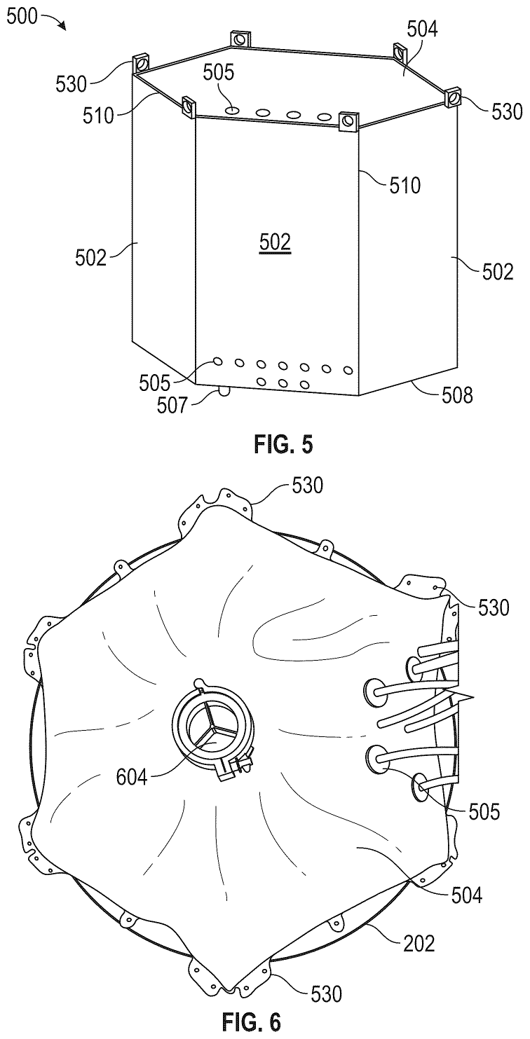

Referring now to FIG. 5, a vessel configured for use with embodiments of the invention is depicted. As shown, suitable vessels for use with embodiments of the invention include a flexible bioprocessing vessel 500 that is hexagonal. More specifically, the vessel includes at least eight flexible panels forming sides, top and bottom of the flexible bioprocessing vessel. That is, the vessel includes six flexible side panels 502, a top panel 504, and a bottom panel 508 located on an opposite end of the hexagonal flexible bioprocessing vessel from the top panel 504. The eight flexible panels are joined together, e.g., welded, to define an interior configured for processing a fluid. In other embodiments, an additional side panel may be included or removed to change the capacity of the vessel.

The panels are joined together at seams 510. As shown, there are vertical seams that join side panels together as well as substantially horizontal seams that join top and bottom panels to the side panels. In embodiments, these seams are formed via thermal welds.

The flexible bioprocessing vessel 500 may further includes a plurality of fluid inputs/connectors/ports 505 for exhaust filtering, sensing, and adding fluids to the interior cavity. In embodiments, the fluid inputs/connectors/ports are located in one or more rows on a front facing flexible panel of the vessel, and on the top panel. As will be appreciated, the vessel includes at least one fluid output 507 for fluid removal.

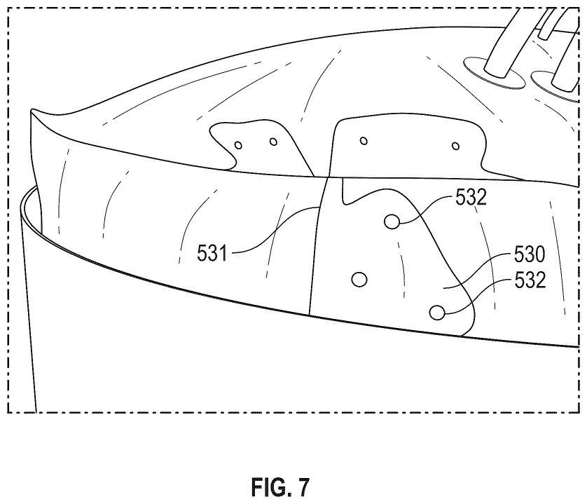

Referring to FIG. 6 and FIG. 7, the vessel includes a plurality of circumferentially arranged hoisting points 530. The hoisting points are located proximate to a junction 531 of each vertical seam and the horizontal seam of the top panel and side panels. The hoisting points may be a section of flexible film that is welded to the vertical and/or horizontal seam of the vessel. In embodiments, the hoisting points are film eyelet tabs that include one or more eyelets 532. The eyelets may be formed as holes in the flexible film. In embodiments, the eyelets may be reinforced with grommets, which may be metal or plastic. The eyelets may be a variety of sizes and are not limited in this regard.

The location of the hoisting points may vary, though, in embodiments, they are spaced about the circumference or perimeter of the vessel proximate to the junction of the vessel side panels and the top panel of the vessel. In embodiments, the hoisting points are evenly spaced about the circumference or perimeter.

Although embodiments of the invention are shown for use with hexagonal bags located within cylindrical support structures, other embodiments may be used with other bag geometries, e.g., octagonal bags.

In a specific embodiment, the flexible bioprocessing vessel has an overall height of about 2100 mm and an overall point to point width of about 1304 mm and is configured for use with a 2000 L rigid support structure having a cylindrical interior.

In another embodiment, the flexible bioprocessing vessel has an overall height of about 1350 mm and an overall point to point width of about 814 mm and is configured for use with a 500 L rigid support structure having a cylindrical interior.

In embodiments, the vessel may be manufactured from a USP Class VI certified material such as, for example, silicone, polycarbonate, polyethylene, and polypropylene. Non- limiting examples of flexible materials include polymers such as polyethylene (for example, linear low-density polyethylene and ultra-low-density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials.

Embodiments of the invention contemplate methods for deploying a flexible bioprocessing vessel. In an initial step, a method includes placing a flexible bioprocessing vessel within a rigid support structure, the flexible bioprocessing vessel having a plurality of circumferentially arranged hoisting points arranged around an upper portion of the flexible bioprocessing vessel. The method further includes connecting the plurality of circumferentially arranged hoisting points to a vessel hoist mechanism. After the hoisting points are connected, the method includes inflating the flexible bioprocessing vessel and hoisting the flexible bioprocessing vessel within the rigid support structure, while it is inflating, via the plurality of circumferentially arranged hoisting points and vessel hoist mechanism until it is deployed within the rigid support structure. The plurality of circumferentially arranged hoisting points reduces a possibility of undesirable wrinkling of the flexible bioprocessing vessel during deployment.

In other embodiments, the hoisting mechanism may be sufficient to deploy a vessel without inflation.

In an embodiment, the flexible bioprocessing vessel includes at least four side panels forming sides of the flexible bioprocessing vessel and a top panel adjoining the at least four side panels, the top panel forming a top of the flexible bioprocessing vessel. The vessel further includes a bottom panel adjoining the at least four side panels, the bottom panel forming a bottom of the flexible bioprocessing vessel, the at least four side panels, top panel, and bottom panel defining an interior cavity configured for processing a fluid, the at least four side panels joined together via vertical welded seams.

In another embodiment, the plurality of circumferentially arranged hoisting points are six circumferentially arranged hoisting points, each of the six circumferentially arranged hoisting points located proximate to a junction of one of the vertical welded seams and a seam connecting the top panel to the at least six side panels.

In an embodiment, the six circumferentially arranged hoisting points are film eyelet tabs.

In an embodiment, the flexible bioprocessing vessel further includes a central hoisting point positioned at an approximate mid-point of a top panel of the flexible bioprocessing vessel.

In an embodiment, the method further includes connecting probes, at least one fluid input, and/or at least one fluid output to the flexible bioprocessing vessel. As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

1. A method for deploying a flexible bioprocessing vessel comprising:
placing a flexible bioprocessing vessel within a rigid support structure, the flexible bioprocessing vessel having a plurality of circumferentially arranged hoisting points arranged around an upper portion of the flexible bioprocessing vessel;
connecting the plurality of circumferentially arranged hoisting points to a vessel hoist mechanism;
inflating the flexible bioprocessing vessel; and
hoisting the flexible bioprocessing vessel within the rigid support structure, while it is inflating, via the plurality of circumferentially arranged hoisting points and vessel hoist mechanism until it is deployed within the rigid support structure;
wherein the plurality of circumferentially arranged hoisting points reduces a possibility of undesirable wrinkling of the flexible bioprocessing vessel during deployment.

2. The method of claim 1 wherein the flexible bioprocessing vessel comprises:
at least six side panels forming sides of the flexible bioprocessing vessel;
a top panel adjoining the at least six side panels, the top panel forming a top of the flexible bioprocessing vessel; and
a bottom panel adjoining the at least six side panels, the bottom panel forming a bottom of the flexible bioprocessing vessel, the at least six side panels, top panel, and bottom panel defining an interior cavity configured for processing a fluid, the at least six side panels joined together via vertical welded seams.

3. The method of claim 2 wherein the plurality of circumferentially arranged hoisting points are six circumferentially arranged hoisting points, each of the six circumferentially arranged hoisting points located proximate to a junction of one of the vertical welded seams and a seam connecting the top panel to the at least six side panels.

4. The method of claim 3 wherein the six circumferentially arranged hoisting points are film eyelet tabs.

5. The method of claim 1 wherein the flexible bioprocessing vessel further includes a central hoisting point positioned at an approximate mid-point of a top panel of the flexible bioprocessing vessel.

6. The method of claim 1 further comprising:
connecting probes, at least one fluid input, and/or at least one fluid output to the flexible bioprocessing vessel.

7. The method of claim 1 wherein the rigid support structure is a 2000 L tank having a cylindrical interior.

8. A flexible bioprocessing vessel comprising:
at least four side panels forming sides of the flexible bioprocessing vessel;
a top panel adjoining the at least four side panels, the top panel forming a top of the flexible bioprocessing vessel;
a bottom panel adjoining the at least four side panels, the bottom panel forming a bottom of the flexible bioprocessing vessel, the at least four side panels, top panel, and bottom panel defining an interior cavity configured for processing a fluid, the at least four side panels joined together via vertical welded seams;
at least one fluid input and at least one fluid output for adding and removing fluid to and from the interior cavity respectively; and
a plurality of circumferentially arranged hoisting points arranged around the top of the flexible bioprocessing vessel;
wherein the plurality of circumferentially arranged hoisting points reduces a possibility of undesirable wrinkling of the flexible bioprocessing vessel during deployment within a rigid support structure.

9. The flexible bioprocessing vessel of claim 8 wherein the at least four side panels are six side panels.

10. The flexible bioprocessing vessel of claim 9 wherein the plurality of circumferentially arranged hoisting points are six circumferentially arranged hoisting points.

11. The flexible bioprocessing vessel of claim 8 wherein the plurality of circumferentially arranged hoisting points are film eyelet tabs.

12. The flexible bioprocessing vessel of claim 8 wherein the flexible bioprocessing vessel further includes a central hoisting point positioned at an approximate mid-point of the top panel of the flexible bioprocessing vessel.

13. The flexible bioprocessing vessel of claim 8 wherein each of the at least four side panels are thermally welded to adjacent side panels.

14. The flexible bioprocessing vessel of claim 8 wherein the at least four side panels are seven side panels.

15. The flexible bioprocessing vessel of claim 8 wherein the at least six side panels are eight side panels.

16. The flexible bioprocessing vessel of claim 9 wherein the flexible bioprocessing vessel has an overall height of about 2100 mm and an overall point to point width of about 1304 mm and is configured for use with a 2000 L rigid support structure having a cylindrical interior.

17. The flexible bioprocessing vessel of claim 9 wherein the flexible bioprocessing vessel has an overall height of about 1350 mm and an overall point to point width of about 814 mm and is configured for use with a 500 L rigid support structure having a cylindrical interior.

18. A system for bioprocessing comprising:
a rigid support structure having a cylindrical interior that includes a bottom surface and a substantially open top;
a vessel hoist mechanism; and
a flexible bioprocessing vessel comprising:
at least four side panels forming sides of the flexible bioprocessing vessel;
a top panel adjoining the at least four side panels, the top panel forming a top of the flexible bioprocessing vessel;
a bottom panel adjoining the at least four side panels, the bottom panel forming a bottom of the flexible bioprocessing vessel, the at least four side panels, top panel, and bottom panel defining an interior cavity configured for processing a fluid, the at least six side panels joined together via vertical welded seams;
at least one fluid input and at least one fluid output for adding and removing fluid to and from the interior cavity respectively; and
a plurality of circumferentially arranged hoisting points arranged around the top of the flexible bioprocessing vessel, the plurality of circumferentially arranged hoisting points configured for attachment to the vessel hoist mechanism;
wherein the plurality of circumferentially arranged hoisting points reduces a possibility of undesirable wrinkling of the flexible bioprocessing vessel during deployment within the rigid support structure.

19. The system of claim 18 wherein the at least four side panels are six side panels.

20. The system of claim 19 wherein the plurality of circumferentially arranged hoisting points are six circumferentially arranged hoisting points, each of the six circumferentially arranged hoisting points.

21. The system of claim 18 wherein the plurality of circumferentially arranged hoisting points are film eyelet tabs.

22. An apparatus for deploying a flexible bioprocessing vessel within a rigid support structure, the apparatus comprising:
a motor support structure that extends over an open top of the rigid support structure;
a motor connected to the motor support structure,
an attachment frame connected to the motor, the attachment frame configured for attachment to a plurality of circumferentially arranged hoisting points arranged around a top panel of the flexible bioprocessing vessel; and
wherein the apparatus is configured to hoist the flexible bioprocessing vessel within the rigid support structure, while it is inflating, via the plurality of circumferentially arranged hoisting points until it is deployed within the rigid support structure;
wherein the apparatus and the plurality of circumferentially arranged hoisting points reduces a possibility of undesirable wrinkling of the flexible bioprocessing vessel during deployment.

23. The apparatus of claim 22 wherein the attachment frame comprises:
a central member which is attached to a selectively retractable line of the motor;
a hoist member formed on or attached to each distal end of the central member; and
a plurality of downwardly depending legs located on each hoist member, the downwardly depending legs configured for connection to the plurality of circumferentially arranged hoisting points arranged around the top of the flexible bioprocessing vessel.

24. The apparatus of claim 22 wherein the attachment frame comprises:
a hub that includes a plurality of radially extending spokes that are configured for connection to the plurality of circumferentially arranged hoisting points arranged around the top of the flexible bioprocessing vessel.
